(19) United States
(12) Patent Application Publication
(10) Pub. No.: US 2025/0115842 A1
(43) Pub. Date: Apr. 10, 2025

(54) SAMPLE PROCESSING CARTRIDGE AND COLLECTION APPARATUS

(71) Applicants: CANON MEDICAL SYSTEMS CORPORATION, Otawara-shi (JP); Canon Kabushiki Kaisha, Tokyo (JP)

(72) Inventor: Shinya SUGIYAMA, Nasushiobara (JP)

(73) Assignees: CANON MEDICAL SYSTEMS CORPORATION, Otawara-shi (JP); Canon Kabushiki Kaisha, Tokyo (JP)

(21) Appl. No.: 18/905,824

(22) Filed: Oct. 3, 2024

(30) Foreign Application Priority Data
Oct. 6, 2023 (JP) 2023-174458

(51) Int. Cl. C12M 3/00; C12M 1/00

(52) U.S. Cl. CPC C12M 23/42; C12M 23/40

(57) ABSTRACT

A sample processing cartridge according to an embodiment includes a closed-system flow channel, a housing container, and a collection mechanism. The closed-system flow channel is a flow channel configured to process a liquid containing a sample. The housing container houses the closed-system flow channel. The collection mechanism is disposed inside the housing container and performs an operation for gathering the closed-system flow channel.

### CROSS-REFERENCE TO RELATED APPLICATIONS

This application is based upon and claims the benefit of priority from Japanese Patent Application No. 2023-174458, filed on Oct. 6, 2023, the entire contents of which are incorporated herein by reference.

### FIELD

Embodiments described herein relate generally to a sample processing cartridge and a collection apparatus.

### BACKGROUND

Conventional closed-system devices, such as a cell culture device including a closed-system flow channel and a cell culture device including a closed-system container, have been known.

### BRIEF DESCRIPTION OF THE DRAWINGS

FIG. 1 is a block diagram illustrating an example of the configuration of a cell culture apparatus according to an embodiment;

FIG. 2 is a diagram for illustrating an example of the configuration of a collection mechanism according to the embodiment;

FIG. 3 is a diagram for illustrating an example of the configuration of the collection mechanism according to the embodiment;

FIG. 4 is a diagram for illustrating an example of the configuration of the collection mechanism according to the embodiment;

FIG. 5 is a diagram for illustrating an example of the configuration of the collection mechanism according to the embodiment;

FIG. 6 is a diagram for illustrating an example of the configuration of an aseptic cutting mechanism according to the embodiment;

FIG. 7 is a diagram for illustrating an example of the screen configuration of a cutting instruction screen according to the embodiment;

FIG. 8 is a flow chart illustrating an example of processing performed by the cell culture apparatus according to the embodiment;

FIG. 9 is a schematic diagram illustrating an example of a cell culture apparatus according to a second modification; and

FIG. 10 is a block diagram illustrating an example of the configuration of a cell culture apparatus according to a third modification.

### DETAILED DESCRIPTION

A sample processing cartridge according to an embodiment includes a closed-system flow channel, a housing container, and a collection mechanism. The closed-system flow channel is a flow channel configured to process a liquid containing a sample. The housing container houses the closed-system flow channel. The collection mechanism is disposed inside the housing container and performs an operation for gathering the closed-system flow channel.

The following describes embodiments of the sample processing cartridge and the collection apparatus in detail with reference to the accompanying drawings. The sample processing cartridge and the collection apparatus according to the present application are not limited by the following embodiments. The embodiments can be combined with other embodiments and conventional technologies to the extent that their contents produce no contradictions. In the following description, the same constituents are denoted by a common symbol, and duplicated descriptions thereof are omitted.

A cell culture apparatus according to the present embodiment includes a double structure container including the housing container configured to house the closed-system flow channel. The double structure container is an example of the sample processing cartridge.

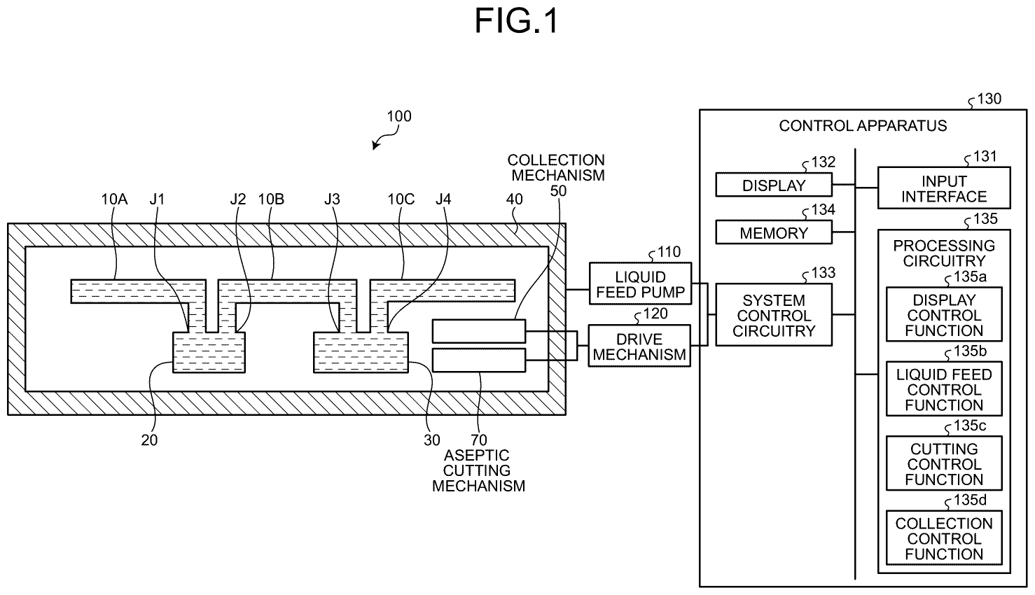

The following describes the details of the cell culture apparatus according to the present embodiment with reference to an example. FIG. 1 is a diagram illustrating an example of a cell culture apparatus 100 according to a first embodiment.

The cell culture apparatus 100 includes closed-system flow channels 10A to 10C, a reagent container 20, a culture container 30, a housing container 40, a collection mechanism 50, an aseptic cutting mechanism 70, a liquid feed pump, a drive mechanism 120, and a control apparatus 130. In the following description, the closed-system flow channels 10A to 10C, the reagent container 20, and the culture container 30 are also referred to as a closed-system device.

The closed-system flow channels 10A to 10C are, for example, pipes configured to feed liquids, for example, reagents such as culture media for use in cell culture and cell suspensions containing target cells to be cultured. The closed-system flow channels 10A to 10C are formed of a flexible material such as silicone or resin, for example. For example, the closed-system flow channels 10A to 10C feed liquids, such as culture media and cell suspensions, from the reagent container 20 to the culture container 30 during a cell culture process such as culture medium exchange. Hereinafter, the closed-system flow channels 10A to 10C are also simply referred to as a closed-system flow channel 10 when not specifically distinguished from each other.

One end of the closed-system flow channel 10A is connected to the reagent container 20 in a communicable manner via a connection J1. The other end of the closed-system flow channel 10A is closed so as not to be in contact with the outside. One end of the closed-system flow channel 10B is connected to the reagent container 20 in a communicable manner via a connection J2. The other end of the closed-system flow channel 10B is connected to the culture container 30 in a communicable manner via a connection J3. One end of the closed-system flow channel 10C is connected to the culture container 30 in a communicable manner via a connection J4. The other end of the closed-system flow channel 10C is closed so as not to be in contact with the outside.

Thus, the closed-system flow channels 10A to 10C, the reagent container 20, and the culture container 30 constitute a single closed-system flow channel as a whole. In other words, the closed-system device is an example of the closed-system flow channel.

The closed end of the closed-system flow channel 10A may include a connection (not illustrated) that can connect the closed-system flow channel 10A and another closed-system flow channel to each other in a communicable manner. For example, as the connection, what is called an aseptic connection joint can be used. In this case, a single closed-system flow channel can be configured, for example, even when the configuration of the flow channels of the cell culture apparatus 100 is changed. The closed-system flow channel 10C may include the same connection as that of the closed-system flow channel 10A.

The reagent container 20 is a container configured to store reagents such as culture media for use in the cell culture process. The reagent container 20 is formed of, for example, a flexible material such as silicone or resin.

The culture container 30 is a container configured to culture a target cell. The culture container 30 is formed of a rigid material such as resin.

FIG. 1 illustrates an example in which the entirety of the closed-system flow channels and the containers are filled with a liquid, but the state of the liquid present in the closed-system flow channels and the containers is not limited to this example. For example, a liquid may be present only in the containers, meanwhile the liquid may be present in the closed-system flow channels only during liquid feeding. The same applies to the other drawings.

The housing container 40 is a container configured to house the closed-system device. By housing the closed-system device in the housing container 40, the cell culture apparatus 100 constitutes a container that houses a cell suspension and the like by a double structure composed of the closed-system device and the housing container 40. Note that the cell culture apparatus 100 may be configured as a multiple structure container by providing the housing container 40 with a double or more complex structure.

In the present embodiment, the housing container 40 is formed of a rigid body such as resin or metal and has a shape, such as a rectangular parallelepiped shape or a cubic shape, that allows the closed-system device to be housed.

The housing container 40 may be openable and closable. When the housing container 40 is openable and closable, a gasket or the like is preferably provided in an opening-and-closing portion in order to prevent the liquid leakage of reagents or samples inside the housing container 40 and the entry of foreign matters from the outside of the housing container 40.

This configuration enables the user to perform manual processing such as taking some of the target cells by using a pipette for the target cells or checking the connection state between the closed-system flow channel 10 and the culture container 30. The housing container 40 may also have a double (multiple) structure for each compartment. For example, in FIG. 1, a double structure may be configured in such a manner that a compartment including the reagent container 20 and the closed-system flow channel 10 connected to the reagent container 20 and a compartment including the culture container 30 and the closed-system flow channel 10 connected to the culture container 30 are separately disposed.

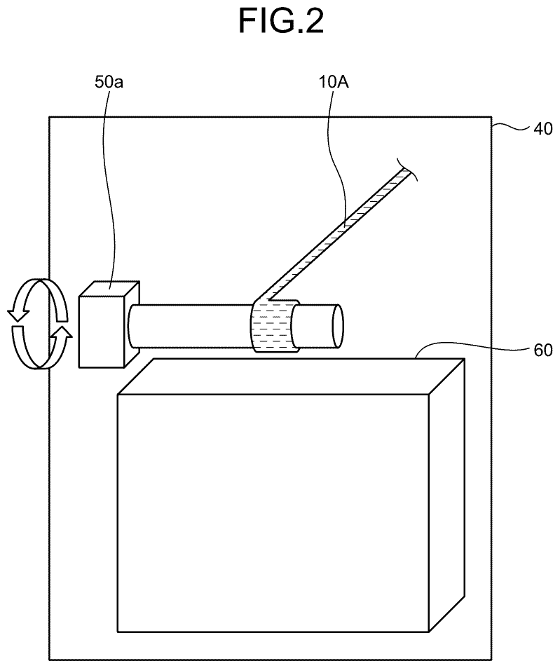

The collection mechanism 50 is disposed inside the housing container 40 and performs an operation for gathering the closed-system device. Furthermore, the collection mechanism 50 moves the gathered closed-system device to a disposal container 60 (see FIG. 2 to FIG. 5). For example, the disposal container 60 is a container configured to collectively dispose of the closed-system device. The disposal container 60 includes an opening for collecting the closed-system device. The disposal container 60 is an example of a collector. Hereinafter, the configuration of the collection mechanism 50 will be described using FIG. 2 to FIG. 5.

First, the collection of the closed-system device formed of a flexible material will be described. FIG. 2 to FIG. 4 are diagrams illustrating an example of the configuration of a collection mechanism 50a. The collection mechanism 50a is configured to wind up and gather the closed-system device in one place to make the closed-system device ready for disposal and thereby collect the closed-system device.

As illustrated in FIG. 2, the disposal container 60 is disposed below the collection mechanism 50a. For example, a used closed-system device is collected into the disposal container 60 and then disposed of by an operator (user) in an appropriate manner, for example, disposed of after sterilization.

The collection mechanism 50a is supported inside the housing container 40 by a not-illustrated support. When the drive mechanism 120 described later is driven, the collection mechanism 50a rotates around the central axis of the collection mechanism 50a as indicated by arrows in FIG. 2. This allows the collection mechanism 50a to wind up and gather the closed-system flow channel 10A in one place. After use, the collection mechanism 50a may be cleaned and sterilized and then reused.

In the example in FIG. 2, the closed-system flow channel 10A is wound up, but the closed-system flow channels 10B and 10C may be wound up. Alternatively, for example, in the case where the reagent container 20 and the culture container 30 are formed of a flexible material and can be gathered in one place together with the closed-system flow channel 10, the collection mechanism 50a may wind up the reagent container 20 and the culture container 30 together with the closed-system flow channel 10.

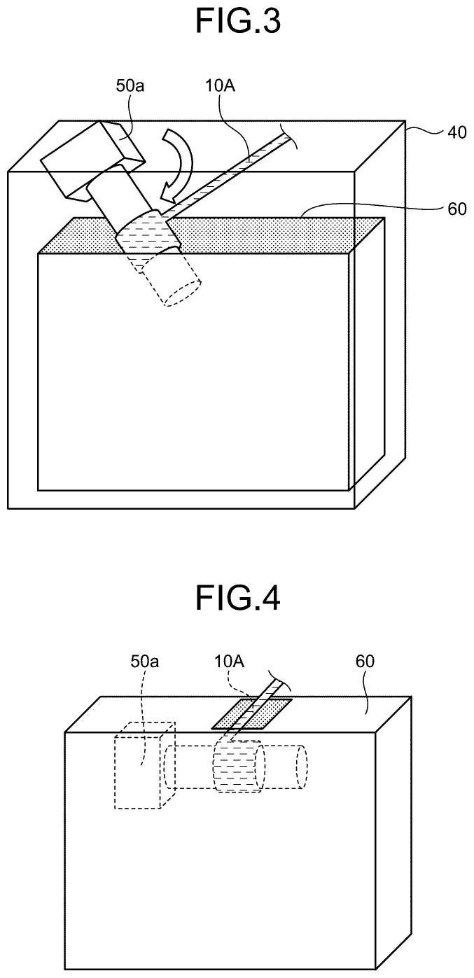

Furthermore, when the collection mechanism 50a itself is inclined as indicated by an arrow in FIG. 3, the collection mechanism 50a drops the closed-system flow channel 10A wound up and gathered in one place toward the opening of the disposal container 60. Thus, the collection mechanism 50a can move the closed-system flow channel 10A gathered in one place into the disposal container 60. Although not illustrated, the collection mechanism 50a may include a mechanism configured to push out the closed-system flow channel 10A gathered in one place toward the disposal container 60.

In the examples in FIG. 2 and FIG. 3, the collection mechanism 50a is disposed outside the disposal container 60, but the collection mechanism 50a may be disposed inside the disposal container 60. FIG. 4 is a diagram illustrating an example of a case in which the collection mechanism 50a is disposed inside the disposal container 60.

In the example in FIG. 4, the collection mechanism 50a is rotatably disposed inside the disposal container 60. Also in the example in FIG. 4, when the drive mechanism 120 is driven, the collection mechanism 50a rotates. The rotation of the collection mechanism 50a causes the closed-system flow channel 10A to pass through the opening of the disposal container 60 and be wound up and gathered in one place by the collection mechanism 50a. When the collection mechanism 50a is disposed inside the disposal container 60, the collection mechanism 50a preferably disposes of the closed-system device gathered in one place.

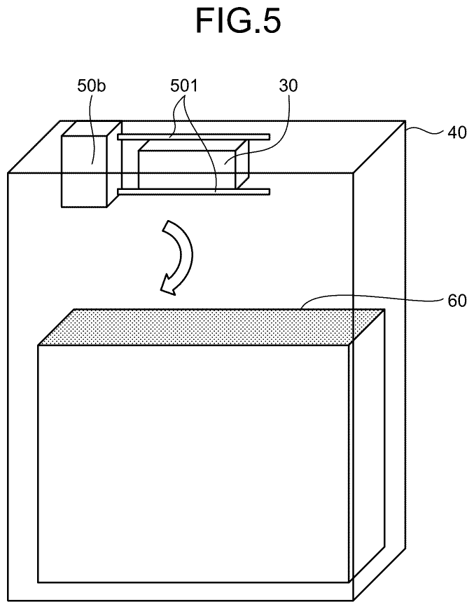

Next, a collection mechanism 50b configured to collect a closed-system device that cannot be gathered in one place will be described. The case in which the closed-system device cannot be gathered in one place is such that, for example, it is difficult to wind up and gather the closed-system device in one place because the closed-system device is formed of a rigid body or for other reasons. FIG. 5 is a diagram illustrating an example of the configuration of the collection mechanism 50b.

As illustrated in FIG. 5, the collection mechanism 50b is disposed near the culture container 30. Furthermore, the disposal container 60 is disposed below the culture container 30. The disposal container 60 includes an opening.

The collection mechanism 50b includes a pair of supports 501. The pair of the supports 501 supports the culture container 30 by sandwiching the culture container 30 therebetween. On receipt of an instruction from the user to collect the culture container 30, the collection mechanism 50b moves via a not-illustrated rail or the like to a position where the culture container 30 can be sandwiched between the pair of the supports 501. Then, when the drive mechanism 120 is driven, the collection mechanism 50b rotates around the central axis of the collection mechanism 50b as indicated by an arrow in FIG. 5 to drop (move) the culture container 30 into the disposal container 60.

In the example in FIG. 5, the collection mechanism 50b collects the culture container 30, but a target to be collected by the collection mechanism 50b is not limited to the culture container 30. For example, in the case where the reagent container 20 is formed of a rigid material or has a shape that makes it difficult to gather the reagent container 20 in one place, the reagent container 20 may be collected by the collection mechanism 50b.

The collection mechanism 50b may include a mechanism configured to adjust the distance between the pair of the supports 501. This mechanism allows the force of sandwiching the closed-system device to be strengthened (locked), whereby the closed-system device can be prevented from being dropped into any place other than the disposal container 60. On the occasion of dropping (moving) the closed-system device into the disposal container 60, the force of sandwiching is loosened, whereby the closed-system device can be surely moved into the disposal container 60.

Referring back to FIG. 1, the description is continued. The aseptic cutting mechanism 70 maintains an aseptic state and cuts the flow channel. For example, the aseptic cutting mechanism 70 cuts the closed-system flow channel 10 by thermocompression bonding near the connections (J1 to J4) between the closed-system flow channel 10 and the reagent container 20 or the culture container 30.

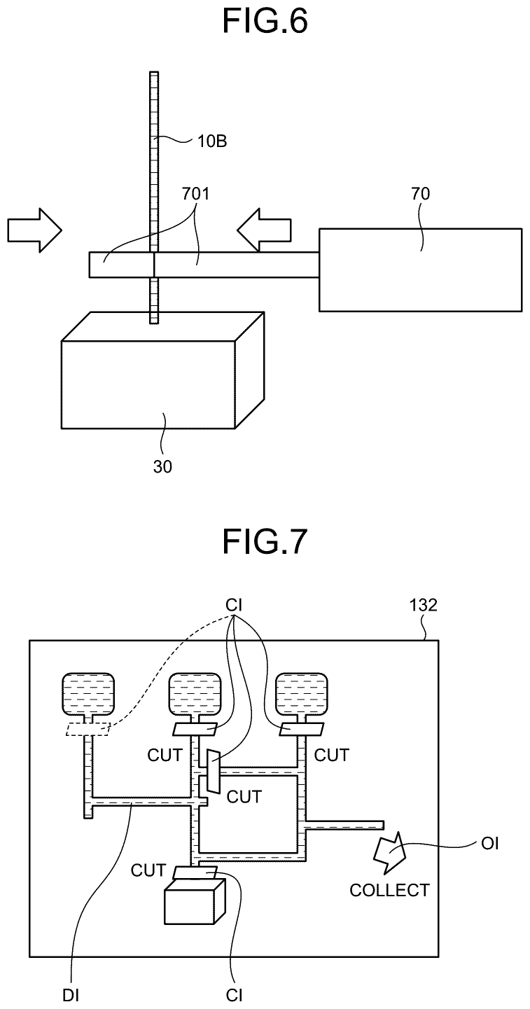

Note that a cutting method is not limited to thermocompression bonding. For example, cutting may be performed by, for example, physically operating the reagent container 20 or the culture container 30 with a motor or the like and thereby disconnecting the closed-system flow channel 10 from the reagent container 20 or the culture container 30. Hereinafter, the aseptic cutting mechanism 70 will be described using FIG. 6. FIG. 6 is a diagram illustrating an example of the configuration of the aseptic cutting mechanism 70.

As illustrated in FIG. 6, the aseptic cutting mechanism 70 includes a pair of cutting units 701. At least one of the paired cutting units 701 includes a heating unit such as a heater. The drive mechanism 120 is driven, whereby the cutting units 701 are driven to sandwich the closed-system flow channel 10B from both sides, as indicated by arrows in FIG. 6. Furthermore, the cutting units 701 heat and apply compress-bonding to a sandwiched portion of the closed-system flow channel 10B. Then, the cutting units 701 fuse the compress-bonded portion by making use of heat emitted by the heating unit.

The above-described heating using the heating unit may be performed, for example, in response to a switch operation by the user, or may be started under the control of a processing circuitry 135 of the control apparatus 130 described later. In the case where the processing circuitry 135 controls the heating unit, the aseptic cutting mechanism 70 is connected to a system control circuitry 133 described below.

In the example in FIG. 6, the aseptic cutting mechanism 70 cuts between the closed-system flow channel 10B and the culture container 30, but a cutting point is not limited to this. For example, the aseptic cutting mechanism 70 may cut between the closed-system flow channel 10A and the reagent container 20.

A liquid feed pump 110 feeds a liquid into the closed-system device. For example, the liquid feed pump 110 is driven under the control of the processing circuitry 135 of the control apparatus 130 to supply, for example, biological samples and reagents such as culture media and cell suspensions into the closed-system device. Likewise, for example, the liquid feed pump 110 also discharges used reagents and the like from the closed-system device.

The drive mechanism 120 drives the collection mechanism 50 and the aseptic cutting mechanism 70. The drive mechanism 120 is, for example, an actuator that converts energy into a mechanical movement. Examples of the actuator include an electric motor that converts electrical energy into mechanical energy. For example, the drive mechanism 120 is driven under the control of the processing circuitry 135 to operate the collection mechanism 50 and the aseptic cutting mechanism 70.

In the case where the cell culture apparatus 100 includes an open/close mechanism, the drive mechanism 120 may operate the open/close mechanism under the control of the processing circuitry 135.

The control apparatus 130 comprehensively controls the cell culture apparatus 100. The control apparatus 130 includes an input interface 131, a display 132, the system control circuitry 133, a memory 134, and the processing circuitry 135.

The input interface 131 receives various input operations from the user, converts the received input operations into electrical signals, and outputs the electrical signals to the processing circuitry 135. For example, the input interface 131 can be realized by a mouse, a keyboard, a trackball, a switch, a button, a joystick, a touchpad configured to perform an input operation when an operation screen is touched, a touchscreen in which a display screen and a touchpad are integrated, a noncontact input circuit using an optical sensor, a voice input circuit, or the like.

The display 132 displays various types of information. For example, the display 132 displays a graphical user interface (GUI) for receiving various commands, settings, and the like from the user via the input interface 131.

The display 132 is, for example, a liquid crystal display, a cathode ray tube (CRT) display, or an organic electro luminescence (EL) display. The display 132 may be a desktop display or may include, for example, a tablet terminal capable of communicating with a main body of the cell culture apparatus 100 by wireless.

The system control circuitry 133 comprehensively controls the operation of the cell culture apparatus 100. For example, the system control circuitry 133 controls the drive of the liquid feed pump 110 and the drive mechanism 120 under the control of the processing circuitry 135.

The memory 134 stores various data. For example, the memory 134 stores various computer programs configured to implement various functions by being read out and executed by the processing circuitry 135. For example, the memory 134 is implemented by a semiconductor memory element such as a random access memory (RAM) or a flash memory, a hard disk, an optical disk, or the like.

The processing circuitry 135 controls the operation of a cell culture apparatus 100B in accordance with an input operation received from an operator via the input interface. For example, the processing circuitry 135 is implemented by a processor.

The processing circuitry 135 reads and executes a computer program stored in the memory 134, and thereby executes a display control function 135a, a liquid feed control function 135b, a cutting control function 135c, and a collection control function 135d.

The display control function 135a controls a display apparatus such as the display 132 to display various types of information on. For example, the display control function 135a causes the display 132 to display a cutting-point selection screen that allows the user to select a point to be cut in the closed-system flow channel 10. Furthermore, for example, the display control function 135a causes the display 132 to display an operation input screen for inputting various types of operation instructions for the cell culture apparatus 100, a setting screen for inputting and displaying a setting for cell culture, and the like.

The liquid feed control function 135b controls the feeding of various types of liquids to be used in the cell culture apparatus 100. For example, the liquid feed control function 135b controls the operation of the liquid feed pump 110 via the system control circuitry 133 to supply liquid samples, liquid reagents (for example, culture media, cell suspensions, and the like), and the like to the closed-system device. Likewise, for example, the liquid feed control function 135b also performs control to discharge used reagents and the like from the closed-system device to a not-illustrated waste bottle.

The cutting control function 135c controls aseptic cutting of the closed-system flow channel 10. For example, the cutting control function 135c receives a selection input from the user via the cutting-point selection screen to select a cutting point in the closed-system flow channel 10. Here, FIG. 7 is a diagram illustrating an example of the screen configuration of a cutting-point selection screen 132a. For example, when the user inputs an operation instruction to cut the closed-system flow channel 10 via the operation input screen, the cutting-point selection screen 132a is displayed on the display 132 by the display control function 135a.

As illustrated in FIG. 7, on the cutting-point selection screen 132a, a closed-system device icon DI, a cutting icon CI, and a collection icon OI are displayed.

The closed-system device icon DI simply represents the configuration of the closed-system flow channel 10, the reagent container 20, and the culture container 30 in the cell culture apparatus 100. An indication by the closed-system device icon DI changes according to how the closed-system flow channel 10, the reagent container 20, and the culture container 30 are arranged in the housing container 40. In other words, since an indication on the display 132 corresponds to the actual arrangement of the flow channel (container), the user can easily specify a cutting point in the closed-system flow channel 10 on the cutting-point selection screen 132a.

The cutting icon CI is an icon that allows the user to specify a cutting point. In the example in FIG. 7, a cutting icon CI indicating an undetermined cutting point is expressed by a dotted line, meanwhile a cutting icon CI indicating a determined cutting point is expressed by a solid line. A word “cut” is displayed in the vicinity of the cutting icon CI to make it easier for the user to find a cutting point.

The collection icon OI is an icon for specifying a place to collect the closed-system device in the case where a plurality of disposal containers 60 is provided in the housing container 40. In the example in FIG. 7, the collection icon OI is expressed by a solid line, but, like the cutting icon CI, in the case where a collection place has not been determined yet, the collection icon OI may be expressed by a dotted line, meanwhile, in the case where a collection point has been determined, the collection icon OI may be expressed by a solid line. Furthermore, a word “collect” is displayed in the vicinity of the collection icon OI to make it easier for the user to find a collection place.

On receipt of an input indicating final determination of a cutting point from the user on the cutting-point selection screen 132a (for example, on receipt of an input made by pressing a final determination input button not illustrated), the cutting control function 135c controls the drive mechanism 120 via the system control circuitry 133 to cut a user-selected point in the closed-system flow channel 10.

Note that, in the present embodiment, the cutting control function 135c is the function of cutting a point specified by the user, but a cutting point is not limited to this. For example, the cutting control function 135c may cut a predetermined point on receipt of an operational input instructing the collection of the closed-system device from the user.

Referring back to FIG. 1, the description is continued. The collection control function 135d controls the collection of the closed-system device. For example, the collection control function 135d receives an input for specifying a closed-system device to be collected, from the user via the operation input screen. The collection control function 135d controls the operation of the drive mechanism 120 via the system control circuitry 133 in accordance with a user input to collect the specified closed-system device.

In the case where the collection control function 135d determines that it is difficult to collect the user-selected closed-system device prior to the start of an operation for collecting the closed-system device (for example, in the case where the number of branches in the closed-system flow channel is equal to or larger than a predetermined number or in the case where a predetermined condition, such as the inclusion of a rigid body, is met), the display control function 135a may cause the display 132 to display a message urging the user to cut the closed-system flow channel 10.

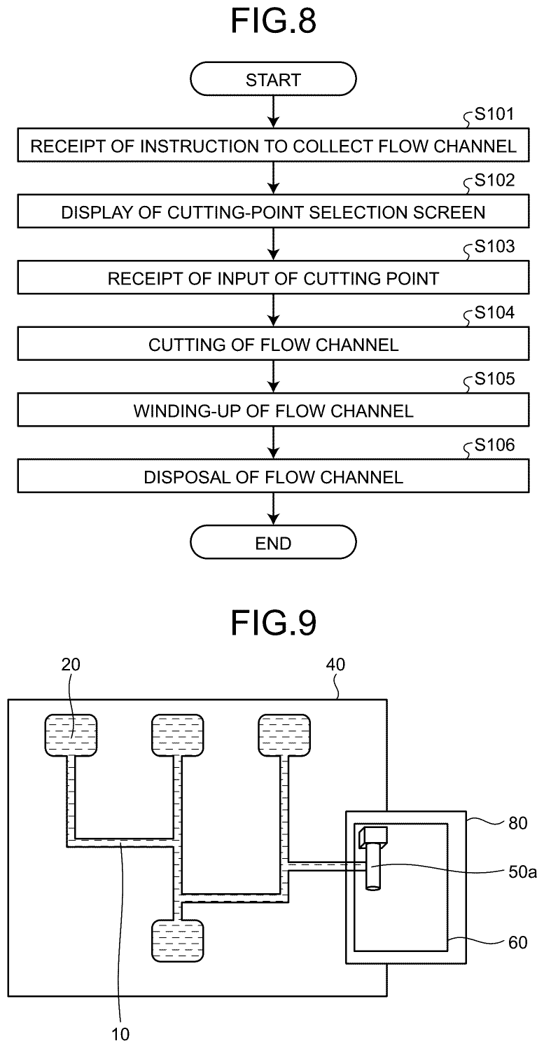

Next, processing performed by the cell culture apparatus 100 will be described. FIG. 8 is a flow chart illustrating an example of the processing performed by the cell culture apparatus 100.

First, the collection control function 135d receives an instruction to collect the closed-system flow channel 10 (step S101). For example, the collection control function 135d receives an operation instruction from the user via the operation input screen displayed on the display 132 to collect the closed-system flow channel 10. In FIG. 8, an example of collecting the closed-system flow channel 10 is described, but the cell culture apparatus 100 may collect the reagent container 20 and the culture container 30.

Next, the display control function 135a causes the display 132 to display the cutting-point selection screen (step S102). Next, the cutting control function 135c receives an input from the user to select a cutting point (step S103). For example, the cutting control function 135c receives an input from the user via the cutting-point selection screen displayed at step S102 to select a cutting point.

Next, the cutting control function 135c cuts the user-selected point in the closed-system flow channel 10 (step S104). For example, the cutting control function 135c controls the drive mechanism 120 via the system control circuitry 133 to cut the point in the closed-system flow channel 10, the point being selected by the user at step S103.

Next, the collection control function 135d winds up and gathers the closed-system flow channel 10 (step S105). For example, the collection control function 135d controls the drive mechanism 120 via the system control circuitry 133 to rotate the collection mechanism 50, whereby the closed-system flow channel 10 cut off from the reagent container 20 or the culture container 30 at step S104 is wound up and gathered in one place.

Next, the collection control function 135d disposes of the closed-system flow channel 10 into the disposal container 60 (step S106), and thus this processing is terminated. For example, the collection control function 135d controls the drive mechanism 120 via the system control circuitry 133 to tilt the collection mechanism 50, whereby the closed-system flow channel 10 gathered in one place at step S105 is dropped into the disposal container 60.

As described above, the cell culture apparatus 100 according to the embodiment drives the collection mechanism 50 to gather the closed-system devices in one place.

Thus, the cell culture apparatus 100 according to the present embodiment can gather the closed-system device in one place in the housing container 40 and move the closed-system device into the disposal container 60. In other words, the cell culture apparatus 100 according to the present embodiment can make the closed-system device ready to be collected while the housing container 40 is kept closed.

Furthermore, the cell culture apparatus 100 according to the present embodiment causes the collection mechanism 50a to rotate, whereby the closed-system device is wound up and gathered in one place. Thus, as long as the closed-system device to be collected is formed of a flexible material, even when the closed-system device has a large surface area, the closed-system device can be easily gathered in one place.

The cell culture apparatus 100 according to the present embodiment supports the closed-system device by sandwiching the closed-system device between a pair of the supports 501 of the collection mechanism 50b and releases the support state of the closed-system flow channel 10 above the opening of the disposal container 60, whereby the closed-system device is moved into the disposal container 60. This allows the closed-system device to be collected, even when the closed-system device cannot be wound up and gathered in one place because the closed-system device is formed of a rigid material or for other reasons.

The cell culture apparatus 100 according to the present embodiment cuts a part of a flow channel constituting the closed-system device before gathering the closed-system device in one place. Thus, even when the closed-system device as a collection target cannot be gathered in one place because the closed-system device includes a rigid body or the flow channel has too many branches or for other reasons, only a part of the closed-system device, the part being enabled to be gathered in one place, can be cut off and collected.

The embodiments described above can also be performed in a modified manner as appropriate by changing a part of the configurations or functions of each apparatus. Thus, hereinafter, modifications according to the embodiments described above will be described as other embodiments. The following mainly describes points that differ from the embodiments described above and omits detailed descriptions of points common to the details already described. The modifications described below may be implemented individually or implemented in combination with the embodiments described above as appropriate.

### First Modification

The embodiments above have described an aspect in which the drive mechanism 120 is controlled to automatically collect the closed-system device. The present modification describes an aspect in which the user manually operates the collection mechanism 50 to collect the closed-system device.

For example, outside the housing container 40, the cell culture apparatus 100 according to the present modification includes a handle for operating the collection mechanism 50a. As an example, the user turns the handle by hand. In this case, the collection mechanism 50a rotates in conjunction with the rotation of the handle to wind up and gather the closed-system device in one place. Thus, the user turns the handle by hand, whereby the closed-system device formed of a flexible material can be wound up and gathered in one place.

The handle for operating the collection mechanism 50a according to the present modification moves up and down through an operation by the user. In this case, the inclination of the collection mechanism 50a changes in accordance with the up-and-down movement of the handle. Thus, the user adjusts the up-and-down movement of the handle, whereby the closed-system device having been wound up and gathered in one place can be moved into the disposal container 60.

In the present modification, in the case where a plurality of the collection mechanisms 50a is provided, handles respectively corresponding to the collection mechanisms 50a shall be provided outside the housing container 40.

For example, outside the housing container 40, the cell culture apparatus 100 according to the present modification includes a handle for operating the collection mechanism 50b. As an example, the user turns the handle by hand. In this case, the collection mechanism 50b moves on a rail or the like in conjunction with the rotation of the handle. Thus, the user turns the handle by hand, whereby the collection mechanism 50b can be moved to a position of the closed-system device as a collection target.

Furthermore, the handle for operating the collection mechanism 50b according to the present modification moves up and down through an operation by the user. In this case, the collection mechanism 50b changes an angle of the pair of the supports 501 in accordance with the up-and-down movement of the handle. Thus, the user adjusts the up-and-down movement of the handle, whereby the closed-system device can be supported by sandwiching the closed-system device between the supports 501.

Furthermore, with the closed-system device supported by the supports 501, the user turns the handle, whereby the closed-system device as a collection target can be moved to above the opening of the disposal container 60. Furthermore, the user adjusts the up-and-down movement of the handle to change the angle of the pair of the supports 501, and this allows the support state of the closed-system device to be released and the closed-system device to be moved into the disposal container 60.

In the present modification, in the case where a plurality of the collection mechanisms 50b is provided, handles respectively corresponding to the collection mechanisms 50b shall be provided outside the housing container 40.

For example, outside the housing container 40, the cell culture apparatus 100 according to the present modification includes a handle for operating the aseptic cutting mechanism 70. As an example, the user turns the handle by hand. In this case, the aseptic cutting mechanism 70 moves on a rail or the like in conjunction with the rotation of the handle. Thus, the user turns the handle by hand, whereby the aseptic cutting mechanism 70 can be moved to a position of the closed-system device as a cutting target.

Furthermore, the handle for operating the collection mechanism 50b according to the present modification moves up and down through an operation by the user. In this case, the distance between a pair of cutting units 701 of the aseptic cutting mechanism 70 changes in accordance with the up-and-down movement of the handle. Thus, the user adjusts the up-and-down movement of the handle, whereby a cutting point in the closed-system device can be cut by thermo-compressing by the pair of the cutting units 701.

In the present modification, a heating unit of the cutting unit 701 shall start heating when the user operates a switch or the like.

According to the present modification, the user can manually perform a collection-related operation of the cell culture apparatus 100, and consequently the processing load on the processing circuitry 135 of the control apparatus 130 can be reduced.

### Second Modification

The embodiments above have described an aspect in which the collection mechanism 50 and the disposal container 60 are disposed inside the housing container 40. The present modification describes an aspect in which, besides the housing container 40, a collector including the collection mechanism 50 and the disposal container 60 is provided as another unit, and the collector is connected to the housing container 40.

FIG. 9 is a schematic diagram illustrating an example of the configuration of a cell culture apparatus 100 according to a second modification. The cell culture apparatus 100 according to the present modification includes a collector 80 as illustrated in FIG. 9. The collector 80 is an example of a collection apparatus.

In the present modification, the housing container 40 includes an opening. The collector 80 is connected to the housing container 40 via the opening of the housing container 40. A gasket or the like is provided to prevent the formation of a gap between the opening of the housing container 40 and the collector 80. In other words, it can be said that the cell culture apparatus 100 according to the present modification has a double closed structure even in the state in which the collector 80 is connected.

The collector 80 includes the collection mechanism 50a and the disposal container 60 inside. The collector 80 further includes an opening on a side on which the collector 80 is connected to the housing container 40. The closed-system device is moved by the collection mechanism 50a from the opening of the collector 80 into the disposal container 60 in the collector 80.

For example, when the drive mechanism 120 is driven, the collection mechanism 50a rotates to wind up and gather the closed-system flow channel 10 and the reagent container 20 in one place inside the collector 80. Furthermore, when the drive mechanism 120 is driven, the inclination of the collection mechanism 50a changes, whereby the closed-system flow channel 10 and the reagent container 20 having been wound up and gathered in one place are moved into the disposal container 60 in the collector 80.

Thus, the closed-system device is collected into the collector 80, whereby the user can appropriately dispose of the closed-system device by simply detaching the collector 80 from the housing container 40.

In the example in FIG. 9, the collection mechanism 50a is provided in the collector 80, but the collection mechanism 50b and the aseptic cutting mechanism 70 may be provided in the collector 80. For example, in the case where the collection mechanism 50b is provided in the collector 80, the housing container 40 is connected to the collector 80 so that the culture container 30 or the like as a collection target fits into the collector 80 by making use of the opening of the collector 80.

The collection mechanism 50b may be configured to be movable to any place inside the housing container 40 by providing a wheel or the like to a support of the collection mechanism 50b. In this case, the collection mechanism 50b moves from a predetermined position inside the collector 80 to a position of the closed-system device as a collection target. The collection mechanism 50b then supports the closed-system device by sandwiching the closed-system device between the pair of the supports 501, and then returns to the inside of the collector 80 and releases the support state above the opening of the disposal container 60 to move the closed-system device into the disposal container 60. Subsequently, the collection mechanism 50b returns to the predetermined position inside the collector 80.

Furthermore, for example, in the case where the aseptic cutting mechanism 70 is disposed inside the collector 80, the housing container 40 is connected to the collector 80 so that a cutting target portion fits into the collector 80 by making use of the opening of the collector 80.

The aseptic cutting mechanism 70 may be configured to be movable to any place inside the housing container 40 by providing a wheel or the like to a support of the aseptic cutting mechanism 70. In this case, the aseptic cutting mechanism 70 moves from a predetermined position inside the collector 80 to the cutting target portion. Then, the aseptic cutting mechanism 70 returns to the predetermined position inside the collector 80 after cutting the cutting target portion.

According to the present modification, by simply disconnecting the housing container 40 from the collector 80, the closed-system device having been gathered in the disposal container 60 inside the collector 80 can be collected. The user can collect the closed-system device without opening the housing container 40, and therefore, in the case of handling an infectious sample, the risk of infection can be further reduced.

### Third Modification

The cell culture apparatus 100 may be configured to detect liquid leakage from the closed-system device and stop an operation for collecting the closed-system device upon the detection of a liquid leak.

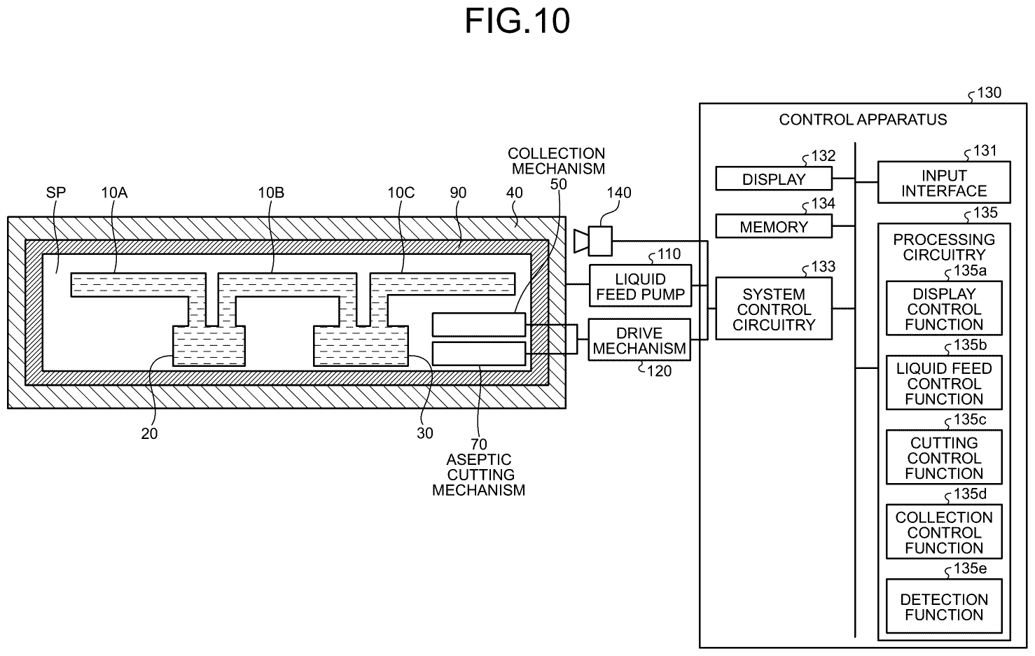

FIG. 10 is a block diagram illustrating an example of the configuration of a cell culture apparatus 100 according to a third modification. The configuration of the cell culture device 100 according to the present modification is approximately the same as that of the cell culture apparatus 100 illustrated in FIG. 1, but is different in that a detection member 90 is provided inside the housing container 40, a camera 140 is provided, and the processing circuitry 135 of the control apparatus 130 includes a detection function 135e.

The detection member 90 is a member configured to detect a liquid present in a detection space SP of the housing container 40. As an example, the detection member 90 is a liquid reagent and is applied to an inner wall of the housing container 40. The detection member 90 is, for example, a liquid reagent having a chemical property that changes on contact with a liquid (for example, an aqueous cobalt chloride solution that reacts with water to change its color).

The detection member 90 may be applied to the entirety of the inner wall of the housing container 40 as illustrated in FIG. 10 or applied only to a partial area (for example, the bottom area of the housing container 40).

The detection member 90 changes its color on contact with a liquid, and therefore, for example, when a liquid leak from the closed-system device into the detection space SP occurs or when a liquid enters the detection space SP from the outside of the housing container 40, the color changes by contact of the liquid with the detection member 90 inside the wall of the housing container 40.

When the housing container 40 has an openable-and-closable structure, there is a possibility that the detection member 90 applied to the inner wall is lost due to friction or the like caused by an opening and closing operation. Therefore, the cell culture apparatus 100 may be configured such that the detection member 90 as a liquid reagent can be reapplied to the inner wall of the housing container 40 by using a spray nozzle or the like each time an opening and closing operation is performed.

In the present modification, the detection space SP is preferably filled with a gas that does not react with the detection member 90. Thus, false detection of the presence of a liquid can be prevented, the false detection being caused by a change in the chemical property of the detection member 90 due to a reaction between the gas present in the detection space SP and the detection member 90.

The camera 140 captures an image of the inside of the housing container 40. For example, the camera 140 is connected to the system control circuitry 133 of the control apparatus 130. The camera 140 captures a camera image of the inside of the housing container 40 under the control of the processing circuitry 135 of the control apparatus 130. The camera 140 transmits the captured camera image to the control apparatus 130.

In the present modification, the camera 140 is provided outside the housing container 40, but the camera 140 may be provided inside the housing container 40. In this case, the camera 140 and the control apparatus 130 may be connected to each other wirelessly or connected in a wired manner. When the camera 140 and the control apparatus 130 are connected to each other in a wired manner, the inside of the housing container 40 is preferably not in contact with the outside of the housing container 40 during the culture of a target cell.

The detection function 135e of the processing circuitry 135 detects a liquid present inside the housing container 40, based on the camera image obtained by capturing the inside of the housing container 40. The presence of a liquid inside the housing container 40 means that there is a possibility of a liquid leak from the closed-system device, hence it can be said that the detection function 135e detects liquid leakage from the closed-system device.

For example, the detection function 135e analyzes a camera image stored in the memory 134, and detects liquid leakage from the closed-system device, based on a visual change in the detection member 90 applied to the inner wall of the housing container 40. Known image analysis techniques can be used for analysis processing on the camera image.

In the present modification, the cutting control function 135c and the collection control function 135d stop operations related to the collection of the closed-system device (such as cutting of the closed-system flow channel 10 and collection of the closed-system device) when a liquid leak from the closed-system device is detected by the detection function 135e.

Here, when an operation related to the collection of the closed-system device is executed on the occurrence of a liquid leak from the closed-system device, the possibility of diffusion of an infectious liquid inside the housing container 40 arises. In the present modification, when a liquid leak from the closed-system device is detected by the detection function 135e, an operation related to the collection of the closed-system device is stopped. Therefore, according to the present modification, an increase in the risk of infection when the user takes out the disposal container 60 from the housing container 40 can be prevented.

The embodiments and the modifications above have described the processing functions of the processing circuitry 135. Here, for example, each of the processing functions described above is stored in the memory 134 in the form of a computer program executable by a computer. The processing circuitry 135 reads the computer programs from the memory 134 and executes the read computer programs to implement the processing functions respectively corresponding to the computer programs. In other words, the processing circuitry 135 having read the computer programs includes the processing functions illustrated in FIG. 1.

In FIG. 1, an example in which the processing functions are implemented by the single processing circuitry 135 is described, but the embodiments are not limited to this example. For example, the processing circuitry 135 may include a combination of a plurality of independent processors, and the processors may execute their computer programs to implement the processing functions. The processing functions of the processing circuitry 135 may be implemented by being distributed in or integrated into a single or a plurality of pieces of processing circuitry as appropriate.

The term “processor” used in the description of the embodiments above means, for example, circuitry, such as a central processing unit (CPU), a graphics processing unit (GPU), an application specific integrated circuit (ASIC), and a programmable logic device (for example, a simple programmable logic device (SPLD), a complex programmable logic device (CPLD), and a field programmable gate array (FPGA)).

Here, instead of storing a computer program in the memory, the computer program may be incorporated directly into the circuitry of the processor. In this case, the processor reads and executes the computer program incorporated into the circuitry to implement the function. Each of the processors according to the present embodiment is not limited to be configured as a single piece of circuitry, but may be configured as one processor formed by combining a plurality of independent pieces of circuitry to implement its function.

According to at least one of the embodiments described above, the closed-system device can be collected with the closed state maintained.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A sample processing cartridge, comprising:
a closed-system flow channel configured to process a liquid containing a sample;
a housing container configured to house the closed-system flow channel; and
a collection mechanism disposed in the housing container and configured to perform an operation for gathering the closed-system flow channel.

2. The sample processing cartridge according to claim 1, wherein
the collection mechanism
includes a collector configured to collect the closed-system flow channel, and
moves the closed-system flow channel gathered through the operation into the collector.

3. The sample processing cartridge according to claim 2, wherein
the collector includes an opening, and
the collection mechanism rotates around a central axis of the collection mechanism to wind up and gather the closed-system flow channel in one place and moves the closed-system flow channel into the collector by making use of the opening.

4. The sample processing cartridge according to claim 3, wherein
the opening is disposed in a surface of the collector, the surface facing an installation surface of the collector, and
the collection mechanism drops the closed-system flow channel wound up and gathered in one place, from above the opening towards the opening and thereby moves the closed-system flow channel into the collector.

5. The sample processing cartridge according to claim 3, wherein the collection mechanism rotates to wind up the closed-system flow channel into the collector through the opening of the collector and thereby moves the closed-system flow channel into the collector.

6. The sample processing cartridge according to claim 2, wherein
the collector includes an opening, the opening being disposed in a surface of the collector, the surface facing an installation surface of the collector,
the collection mechanism
includes a pair of supports configured to support the closed-system flow channel by sandwiching the closed-system flow channel between the supports, and
releases a support state of the closed-system flow channel above the opening to drop the closed-system flow channel toward the opening, and thereby moves the closed-system flow channel into the collector.

7. The sample processing cartridge according to claim 1, further comprising:
a cutting unit configured to aseptically cut the closed-system flow channel at a predetermined position, wherein
the collection mechanism performs an operation for gathering the closed-system flow channel cut off.

8. The sample processing cartridge according to claim 7, wherein the predetermined position is a position on the closed-system flow channel, the position being specified by a user.

9. A collection apparatus connected to a sample processing cartridge, the sample processing cartridge including: a closed-system flow channel configured to process a liquid containing a sample; and a housing container configured to house the closed-system flow channel, the collection apparatus comprising:
a connection configured to connect the housing container to the collection apparatus in a manner that an interior of the housing container is not in contact with an exterior of the housing container; and
a collection mechanism configured to perform an operation for gathering the closed-system flow channel.
